(19) United States
(12) Patent Application Publication
(10) Pub. No.: US 2020/0161510 A1
(43) Pub. Date: May 21, 2020

(54) LED LIGHTING APPARATUS HAVING STERILIZING FUNCTION

(71) Applicant: SEOUL VIOSYS CO., LTD., Ansan-si (KR)

(72) Inventor: Sang Wook JUNG, Ansan-si (KR)

(21) Appl. No.: 16/686,635

(22) Filed: Nov. 18, 2019

Related U.S. Application Data
(60) Provisional application No. 62/769,142, filed on Nov. 19, 2018.

(51) Int. Cl. H01L 33/50; H01L 33/58; A61L 2/10

(52) U.S. Cl. CPC H01L 33/504; A61L 2202/11; A61L 2/10; H01L 33/58

(57) ABSTRACT

A lighting apparatus including a white light emitting device including at least one first light emitting diode configured to emit white light, and at least one second light emitting diode emitting light configured to emit light having a central wavelength of about 430 nm to sterilize at least one pathogenic microorganism, in which the lighting apparatus is configured to emit white light from the white light emitting device and light from the second light emitting diode to the outside.

### CROSS-REFERENCE TO RELATED APPLICATIONS

This application claims the benefit of U.S. Provisional Application No. 62/769,142, filed on Nov. 19, 2018, which is hereby incorporated in its entirety by reference for all purposes as set forth herein.

### BACKGROUND

### Field

Exemplary embodiments of the invention relate to a lighting apparatus having a sterilizing function using LEDs.

### Discussion of the Background

As an inorganic light source, light emitting diodes have been used in various fields, such as displays, vehicular lamps, general lighting, and the like. With various advantages of the light emitting diodes, such as long lifespan, low power consumption, and rapid response, light emitting diodes have been replacing existing light sources.

Meanwhile, sunlight exhibits a broad spectrum of wavelengths in the ultraviolet, visible, and infrared regions. Further, it is well known in the art that ultraviolet rays have a sterilizing function. Accordingly, various light sources having the sterilizing function using ultraviolet LEDs have been developed.

However, ultraviolet rays with the sterilizing function are generally harmful to the human body, particularly to the eyes or skin of human. As such, light sources using ultraviolet LEDs are subject to restrictions, such as the usage thereof must take place in a space without people. In particular, ultraviolet LEDs with the sterilizing function are not typically suitable for use in lighting apparatuses in places where people are active.

The above information disclosed in this Background section is only for understanding of the background of the inventive concepts, and, therefore, it may contain information that does not constitute prior art.

### SUMMARY

Lighting apparatuses constructed according to exemplary embodiments of the invention are capable of providing a sterilizing function without harming the human body, or without causing eye diseases or dizziness.

Additional features of the inventive concepts will be set forth in the description which follows, and in part will be apparent from the description, or may be learned by practice of the inventive concepts.

A lighting apparatus according to an exemplary embodiment includes a white light emitting device including at least one first light emitting diode configured to emit white light, and at least one second light emitting diode emitting light configured to emit light having a central wavelength of about 430 nm to sterilize at least one pathogenic microorganism, in which the lighting apparatus is configured to emit white light from the white light emitting device and light from the second light emitting diode to the outside.

White light and light emitted from the second light emitting diode may be mixed to be emitted to the outside.

The white light emitting device may include a wavelength converter to convert a wavelength of light emitted from the first light emitting diode.

The wavelength converter may include at least one of a blue phosphor, a green phosphor, and a red phosphor.

Light emitted from the second light emitting diode may be emitted to the outside without passing through the wavelength converter.

The first light emitting diode may be configured to emit blue light having a longer wavelength than that of the second light emitting diode.

The first light emitting diode may be configured to emit light having a central wavelength in a range of 300 nm to 440 nm.

Light generated by the at least one second light emitting diode and emitted to the outside may have a first irradiance, and light generated in the at least one first light emitting diode and emitted to the outside without wavelength conversion may have a second irradiance less than the first irradiance.

The first light emitting diode may be configured to emit light having a central wavelength in a range of 400 nm to 420 nm.

Light generated by the at least one second light emitting diode and emitted to the outside may have a first irradiance, and light generated in the at least one first light emitting diode and emitted to the outside without wavelength conversion may have a second irradiance less than the first irradiance.

The lighting apparatus may further include a circuit board to which the first light emitting diode and the second light emitting diode are mounted.

A lighting apparatus according to another exemplary embodiment includes at least one first light emitting diode, at least one second light emitting diode configured to emit light having a central wavelength of about 430 nm, and a wavelength converter to convert a wavelength of light emitted from the first light emitting diode, in which the first light emitting diode is configured to emit light having a wavelength different from that of the second light emitting diode, and light generated by the second light emitting diode and emitted to the outside has a first irradiance, and light generated in the first light emitting diode and emitted to the outside has a second irradiance less than the first irradiance.

Light emitted from the first light emitting diode may have a central wavelength in a range of 300 nm to 420 nm.

Light wavelength-converted by the wavelength converter and light emitted from the second light emitting diode may be mixed and emitted to the outside.

The mixed light may be white light.

The lighting apparatus may further include a diffusion plate to mix light wavelength-converted by the wavelength converter and light emitted from the second light emitting diode.

The wavelength converter may include at least one of a blue phosphor, a green phosphor, and a red phosphor.

The lighting apparatus may further include a circuit board on which the first and second light emitting diodes are mounted

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the inventive concept, and are incorporated in and constitute a part of this specification, illustrate exemplary embodiments of the inventive concept, and, together with the description, serve to explain principles of the inventive concept.

FIG. 1 is a schematic plan view of a lighting apparatus according to an exemplary embodiment.

FIG. 2 is a schematic cross-sectional view of FIG. 1.

FIG. 3 is a schematic cross-sectional view of a lighting apparatus according to another exemplary embodiment.

FIG. 4 is a schematic cross-sectional view of a lighting apparatus according to another exemplary embodiment.

FIG. 5 is a schematic cross-sectional view of a lighting apparatus according to another exemplary embodiment.

FIG. 6 is a graph illustrating the result of sterilization experiment on pathogenic microorganisms according to wavelengths in a white lighting device.

FIG. 7 is a graph illustrating the result of sterilization experiment on Gram-negative bacteria and Escherichia coli, according to wavelengths of sterilizing light sources.

FIG. 8 is a graph illustrating the result of sterilization experiment on Gram-positive bacteria and Staphylococcus aureus, according to wavelengths of sterilizing light sources.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalties between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z—axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Hereinafter, exemplary embodiments of the present disclosure will be described in detail with reference to the accompanying drawings.

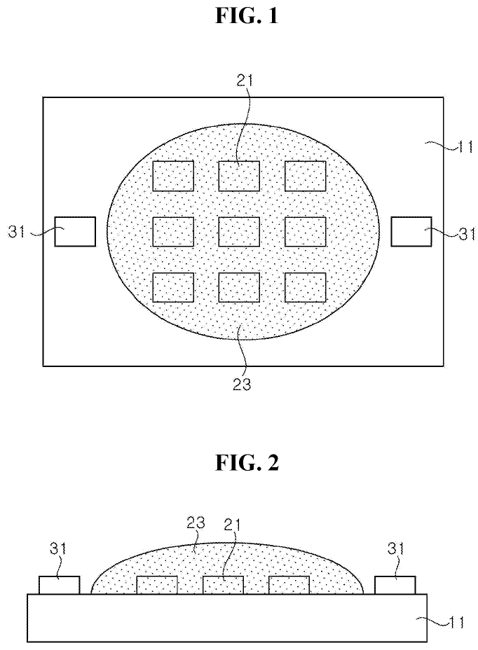

FIG. 1 is a schematic plan view of a lighting apparatus according to an exemplary embodiment, and FIG. 2 is a schematic cross-sectional view of FIG. 1.

Referring to FIG. 1 and FIG. 2, the lighting apparatus may include a circuit board 11, a first light emitting diode 21, a wavelength converter 23, and a second light emitting diode 31.

The circuit board 11 may have a circuit pattern for supplying power to the first and second light emitting diodes 21 and 31. The circuit board 11 may be a printed circuit board, for example, a metal-PCB.

At least one first light emitting diode 21 is mounted on the circuit board 11 as a light source for implementing white light. A plurality of first light emitting diodes 21 may be electrically connected to one another in various ways, for example, in series, in parallel, or in series-parallel.

The first light emitting diode 21 may be, for example, an ultraviolet light emitting diode, a violet light emitting diode, or a blue light emitting diode. The first light emitting diode may be, for example, a light emitting diode having a central wavelength in a range of 300 nm to 420 nm, or may be a blue light emitting diode having a central wavelength in a range of 440 nm to 470 nm.

The wavelength converter 23 converts a wavelength of light emitted from the first light emitting diode 21. The wavelength converter 23 may be, for example, a molding member including a phosphor or a quantum dot. The wavelength converter 23 may cover the first light emitting diode 21. When the plurality of first light emitting diodes 21 are mounted on the circuit board 11, the wavelength converter 23 may cover each of the plurality of first light emitting diodes 21.

The wavelength converter 23 includes a wavelength conversion substance for implementing white light together with light of the first light emitting diode 21. For example, when the first light emitting diode 21 is a blue light emitting diode, the wavelength converter 23 may include a yellow phosphor, or a green phosphor and a red phosphor, or a yellow phosphor and a red phosphor. In this manner, white light may be implemented by mixing blue light emitted from the first light emitting diode 21 with light emitted from the phosphors.

When the first light emitting diode 21 has the central wavelength in a range of 300 nm to 420 nm, the wavelength converter 23 may include a blue phosphor, a green phosphor, and a red phosphor.

The white light may be implemented by the combination of the first light emitting diode 21 and the wavelength converter 23. When the first light emitting diode 21 emits ultraviolet rays, most of the ultraviolet rays are wavelength-converted by the wavelength converter 23, thereby preventing the ultraviolet rays from being emitted to the outside.

According to the illustrated exemplary embodiment, the first light emitting diode 21 and the wavelength converter 23 form a white light emitting device, and white light is implemented through the first light emitting diode 21 and the wavelength converter 23.

The second light emitting diode 31 may be spaced apart from the wavelength converter 23 and be mounted on the circuit board 11. Light emitted from the second light emitting diode 31 may be emitted to the outside without actually entering the wavelength converter 23. Accordingly, the irradiance of the second light emitting diode 31 may be improved.

The second light emitting diode 31 may be connected to the first light emitting diode 21 in series or in parallel, or may be driven independently from the first light emitting diode 21.

The second light emitting diode 31 emits light suitable for sterilizing pathogenic microorganisms other than white light. The second light emitting diode 31 may emit light having a central wavelength of, for example, about 430 nm. In general, while ultraviolet light has a good sterilization effect, ultraviolet light may not be used indoors or in public places where people are active as being harmful to the human body. In addition, visible light of a short wavelength in a range of 400 nm to 420 nm is known to be highly hazardous to the human body. Furthermore, blue light in a range of 440 nm to 450 nm may cause eye diseases and dizziness. On the other hand, light having a wavelength of about 430 nm has a relatively weak problem of ultraviolet light or blue light described above, and also has a sterilization function similar to that at a wavelength to 405 nm, and thus, is capable of being used in a lighting apparatus. As used herein, sterilization may refer to killing or damaging a pathogenic microorganism so as to reduce or prevent the growth of the pathogenic microorganism.

According to the illustrated exemplary embodiment, to add the sterilizing function to the lighting apparatus, the irradiance of light emitted from the second light emitting diode 31 may be greater than that of light emitted from the white light emitting device at the same wavelength. Furthermore, the irradiance of light emitted from the second light emitting diode 31 may be greater than that of light emitted from the first light emitting diode 21 having the central wavelength in the range of 300 nm to 420 nm, which is emitted to the outside of the lighting apparatus. Accordingly, in the lighting apparatus according to the illustrated exemplary embodiment, the sterilizing function is substantially provided by the second light emitting diode 31, as compared to the first light emitting diode 21.

According to an exemplary embodiment, a driving time of the second light emitting diode 31 and the first light emitting diode 21 may be substantially the same, however, the inventive concepts are not limited thereto. In some exemplary embodiments, the driving time of the second light emitting diode 31 may be adjusted according to an installation location of the lighting apparatus. For example, the operating time of the second light emitting diode 31 or a magnitude of the irradiance thereof may be adjusted in consideration of potential hazard to the human body. In this manner, the lighting apparatus according to an exemplary embodiment may prevent the human body from being harmed by light emitted from the second light emitting diode 31.

In this manner, the lighting apparatus according to the illustrated exemplary embodiment is capable of sterilizing pathogenic microorganisms not only in the indoor living space, but also in a space where a large number of people are active, such as an airport or a hospital, thereby preventing human infection by the pathogenic microorganisms.

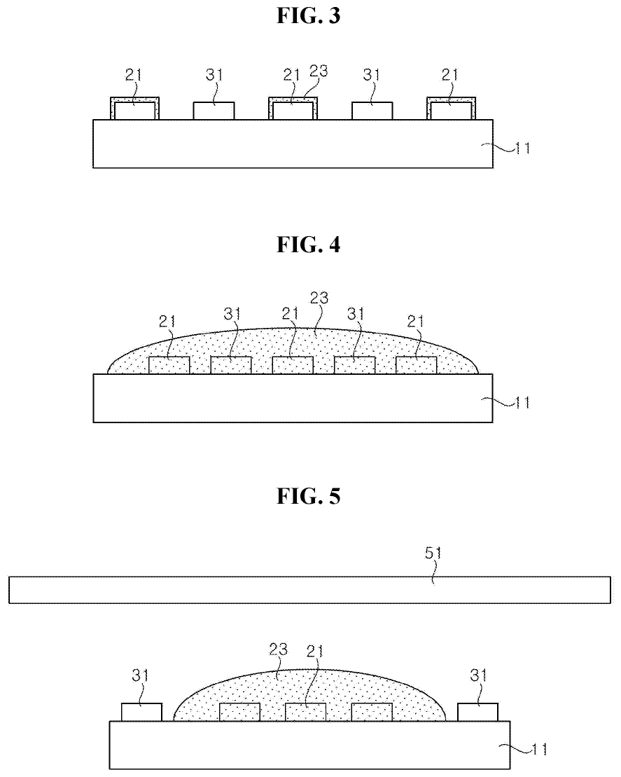

FIG. 3 is a schematic cross-sectional view of a lighting apparatus according to another exemplary embodiment.

Referring to FIG. 3, the lighting apparatus according to the illustrated exemplary embodiment is generally similar to the lighting apparatus described with reference to FIG. 1 and FIG. 2, except that wavelength converters 23 are formed on the first light emitting diodes 21, respectively. More particularly, the wavelength converter 23 shown in FIG. 1 and FIG. 2 covers the entire first light emitting diodes 21, however, according to the illustrated exemplary embodiment, each of the first light emitting diodes 21 is individually covered with the wavelength converter 23.

The wavelength conversion substances in the first light emitting diode 21 and the wavelength converter 23 are substantially the same as those described above, and thus, repeated descriptions thereof will be omitted to avoid redundancy.

As the first light emitting diodes 21 are respectively covered with the wavelength converters 23, the second light emitting diode 31 may be disposed between the first light emitting diodes 21. More particularly, the second light emitting diodes 31 may be uniformly disposed between the first light emitting diodes 21, and thus, light emitted from the second light emitting diode 31 may be mixed with white light. As such, the lighting apparatus according to the illustrated exemplary embodiment may mitigate light emitted from the second light emitting diode 31 from being recognized from the outside.

The second light emitting diodes 31 may be connected in series or in parallel to the first light emitting diodes 21, however, the inventive concepts are not limited thereto. For example, in some exemplary embodiments, the second light emitting diodes 31 may be mounted on the circuit board 11 to be driven independently from the first light emitting diodes 21.

FIG. 4 is a schematic cross-sectional view of a lighting apparatus according to another exemplary embodiment.

Referring to FIG. 4, the lighting apparatus according to the illustrated exemplary embodiment is generally similar to the lighting apparatus described with reference to FIG. 1 and FIG. 2, except that the second light emitting diode 31 is also covered with the wavelength converter 23.

More particularly, the wavelength converter 23 according to the illustrated exemplary embodiment covers not only the first light emitting diode 21, but also the second light emitting diode 31. Accordingly, the wavelength converter 23 may wavelength-convert a portion of light emitted from the second light emitting diode 31.

In this case, since the portion of light emitted from the second light emitting diode 31 is wavelength-converted by the wavelength converter 23, a greater number of second light emitting diodes 31 may be used to implement the irradiance suitable for sterilization, as compared to those described above with reference to FIGS. 1 to 3. Since a fraction of light generated by the second light emitting diode 31 is wavelength-converted to implement white light, the number of the first light emitting diodes 21 may be reduced.

The second light emitting diodes 31 may be uniformly disposed between the first light emitting diodes 21, and thus, uniform light may be emitted to the outside. However, the inventive concepts are not limited thereto.

When the first light emitting diode 21 emits light having the central wavelength in the range of 300 nm to 420 nm, the number and the intensity of the second light emitting diodes 31 may be adjusted, so that the irradiance of light, which is generated by the second light emitting diodes 31 and emitted to the outside without wavelength conversion, is greater than that of light generated in the first light emitting diodes 21 and emitted to the outside without wavelength conversion.

Accordingly, the lighting apparatus according to the illustrated exemplary embodiment also provides an effective sterilizing function by the second light emitting diode 31.

FIG. 5 is a schematic cross-sectional view of a lighting apparatus according to another exemplary embodiment.

Referring to FIG. 5, the lighting apparatus according to the illustrated exemplary embodiment is generally similar to the lighting apparatus described with reference to FIG. 1 and FIG. 2, except that it further includes a diffusion plate 51.

The diffusion plate 51 may increase uniformity of light by mixing white light and light emitted from the second light emitting diode 31. Accordingly, visibility of light emitted from the second light emitting diode 31 may be reduced.

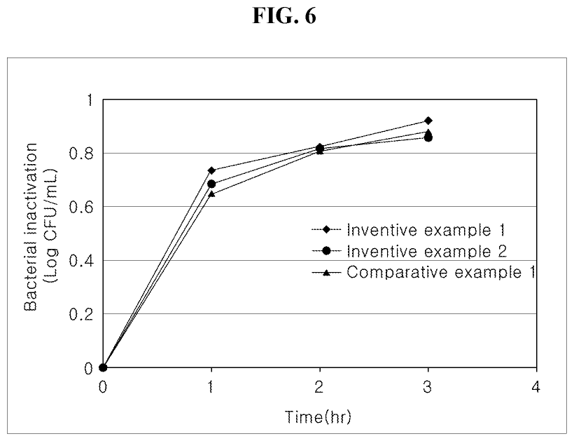

FIG. 6 is a graph illustrating the result of sterilization experiment on pathogenic microorganisms according to wavelengths in a white lighting device.

Sterilization performances on Escherichia coli (E. coli), which is a pathogen, were compared by combining a white light source with a light emitting diode that emits light suitable for sterilization. The white light source and the sterilizing light source (e.g., a second light emitting diode) were irradiated 2 cm away from the medium, in which Gram-negative bacteria, E. coli, was cultured, and the irradiance of the sterilizing light source was 5 mW/cm².

In this experiment, Inventive example 1 is a lighting apparatus including a white light source, which includes a violet light emitting diode having a central wavelength of about 410 nm and a phosphor, and a light emitting diode having a central wavelength of about 430 nm for sterilization. Inventive example 2 is a lighting apparatus including a white light source, which includes a blue light emitting diode having a central wavelength of about 465 nm and a phosphor, and a light emitting diode having the central wavelength of about 430 nm for sterilization. Comparative example 1 is a lighting apparatus including a white light source, which includes a blue light emitting diode having the central wavelength of about 465 nm and a phosphor, and a light emitting diode having a central wavelength of about 405 nm for sterilization.

Referring to FIG. 6, the light source of Inventive example 1 exhibited a relatively favorable sterilizing function, and Inventive example 2 exhibited a sterilizing function of about the same degree as that of the Comparative example 1.

According to the experimental results shown in the graph of FIG. 6, it can be seen that the lighting apparatus emitting visible light of a short wavelength, such as the violet light emitting diode, is more effective for sterilization than that using the blue light emitting diode as the white light source.

While the lighting apparatus of the Comparative example 1 using the short wavelength light of 405 nm shows slightly better performance than Inventive example 2 over time, since the difference therebetween is small, and light of 405 nm is known in the art to be harmful to the human body, the lighting apparatus of the Inventive example 2 may be more suitable for general lighting apparatuses than that of the Comparative example 1.

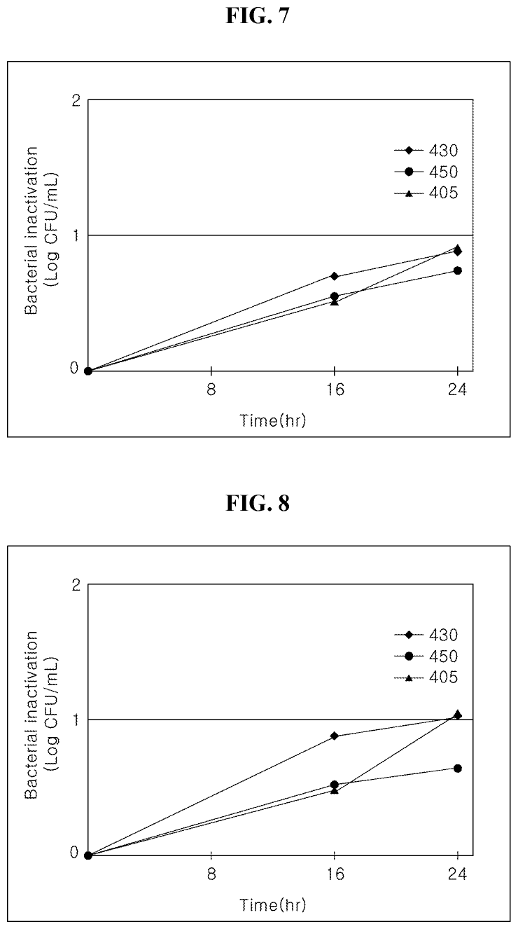

FIG. 7 and FIG. 8 are graphs illustrating the results of sterilization experiment on Gram-negative bacteria, E. coli, and Gram-positive bacteria, Staphylococcus aureus (S. aureus) according to wavelengths of sterilizing light sources, respectively.

Light emitting diodes having peak wavelengths of 405 nm, 430 nm, and 450 nm were used as the sterilizing light sources, respectively, and sterilization performances on E. coli and S. aureus were compared, as shown in FIGS. 7 and 8, respectively. The sterilizing light sources were irradiated 2 m away from the mediums, in which E. coli and S. aureus were cultured, respectively, and the irradiation of sterilizing light sources of 405 nm, 430 nm, and 450 nm were 0.63 mW/cm², 0.81 mW/cm², and 0.81 mW/cm², respectively.

Referring to FIG. 7 and FIG. 8, the sterilizing light source of 430 nm exhibited a favorable sterilization performance compared to that of 450 nm under the same irradiance, and also exhibited a sterilization performance similar to that of 405 nm under 24-hour continuous emission.

In addition, it can also be confirmed that sterilization performances to the E. coli and S. aureus were substantially same, and, accordingly, it can be confirmed that the sterilizing light source of 430 nm wavelength can be suitably used for sterilization of Gram-negative or Gram-positive pathogenic microorganisms.

In the above-described exemplary embodiments, although the first light emitting diode and the second light emitting diode are described as being directly mounted on the circuit board 11, the inventive concepts are not limited thereto. For example, in some exemplary embodiments, at least one of the first light emitting diode and the second light emitting diode may be packaged, and the packages may be mounted on the circuit board 11. Further, in some exemplary embodiments, the package, on which the first light emitting diodes 21 are mounted, may include the wavelength converter 23.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A lighting apparatus, comprising:
a white light emitting device including at least one first light emitting diode configured to emit white light; and
at least one second light emitting diode emitting light configured to emit light having a central wavelength of about 430 nm to sterilize at least one pathogenic microorganism,
wherein the lighting apparatus is configured to emit white light from the white light emitting device and light from the second light emitting diode to the outside.

2. The lighting apparatus of claim 1, wherein white light and light emitted from the second light emitting diode are mixed to be emitted to the outside.

3. The lighting apparatus of claim 1, wherein the white light emitting device comprises a wavelength converter to convert a wavelength of light emitted from the first light emitting diode.

4. The lighting apparatus of claim 3, wherein the wavelength converter comprises at least one of a blue phosphor, a green phosphor, and a red phosphor.

5. The lighting apparatus of claim 3, wherein light emitted from the second light emitting diode is emitted to the outside without passing through the wavelength converter.

6. The lighting apparatus of claim 1, wherein the first light emitting diode is configured to emit blue light having a longer wavelength than that of the second light emitting diode.

7. The lighting apparatus of claim 6, wherein the first light emitting diode is configured to emit light having a central wavelength in a range of 300 nm to 440 nm.

8. The lighting apparatus of claim 7, wherein:
light generated by the at least one second light emitting diode and emitted to the outside has a first irradiance; and
light generated in the at least one first light emitting diode and emitted to the outside without wavelength conversion has a second irradiance less than the first irradiance.

9. The lighting apparatus of claim 7, wherein the first light emitting diode is configured to emit light having a central wavelength in a range of 400 nm to 420 nm.

10. The lighting apparatus of claim 9, wherein:
light generated by the at least one second light emitting diode and emitted to the outside has a first irradiance; and
light generated in the at least one first light emitting diode and emitted to the outside without wavelength conversion has a second irradiance less than the first irradiance.

11. The lighting apparatus of claim 1, further comprising a circuit board to which the first light emitting diode and the second light emitting diode are mounted.

12. A lighting apparatus, comprising:
at least one first light emitting diode;
at least one second light emitting diode configured to emit light having a central wavelength of about 430 nm; and
a wavelength converter to convert a wavelength of light emitted from the first light emitting diode,
wherein the first light emitting diode is configured to emit light having a wavelength different from that of the second light emitting diode, and
wherein light generated by the second light emitting diode and emitted to the outside has a first irradiance, and light generated in the first light emitting diode and emitted to the outside has a second irradiance less than the first irradiance.

13. The lighting apparatus of claim 12, wherein light emitted from the first light emitting diode has a central wavelength in a range of 300 nm to 420 nm.

14. The lighting apparatus of claim 12, wherein light wavelength-converted by the wavelength converter and light emitted from the second light emitting diode are mixed and emitted to the outside.

15. The lighting apparatus of claim 14, wherein the mixed light is white light.

16. The lighting apparatus of claim 12, further comprising a diffusion plate to mix light wavelength-converted by the wavelength converter and light emitted from the second light emitting diode.

17. The lighting apparatus of claim 12, wherein the wavelength converter comprises at least one of a blue phosphor, a green phosphor, and a red phosphor.

18. The lighting apparatus of claim 11, further comprising a circuit board on which the first and second light emitting diodes are mounted.
